(19) United States
(12) Patent Application Publication
(10) Pub. No.: US 2011/0060221 A1
(43) Pub. Date: Mar. 10, 2011

(54) Temperature prediction using medical diagnostic ultrasound

(75) Inventors: Liexiang Fan, Sammamish, WA (US); Kevin Sekins, Yarrow Point, WA (US)

(73) Assignee: Siemens Medical Solutions USA, Inc., Malvern, PA (US)

(21) Appl. No.: 12/554,749

(22) Filed: Sep. 4, 2009

(51) Int. Cl. A61B 8/00

(52) U.S. Cl. 600/438

(57) ABSTRACT

Temperature related information or a temperature characteristic is detected, at least in part, with a medical diagnostic ultrasound system. Anatomy information from an ultrasound scan is used with modeling to determine the temperature or other temperature related parameter. Ultrasound information may be obtained in real-time with application of thermal therapy, so may be used to better control thermal treatment. The anatomy information may be used to align model features measured from a region. The anatomy information may be used as an input into the model. The anatomy information may be used to select an appropriate model, such as selection based on the type of tissue. The anatomy information may be used to correct an output of the model, such as accounting for temperature distribution due to an adjacent vessel.

### BACKGROUND

The present invention relates to determining interior temperature for medical treatment. Thermal energy-based treatments apply heat within a patient. Various modalities, such as RF ablation, microwave, laser irradiation, or high intensity focused ultrasound (HIFU), deliver energy. The safety and efficacy of these treatments are closely correlated with both the end-of-dose tissue temperatures and the time-temperature history of the treated tissue. Time-temperature history is quantified as “thermal dose.”

The temperature and dose are monitored using invasive sensors, such as needle probes. Invasive procedures may be undesired. Magnetic resonance imaging (MRI) monitoring is a noninvasive method to measure tissue treatment temperatures. MRI approaches may not provide real-time feedback and/or are expensive.

### BRIEF SUMMARY

The present invention is defined by the following claims, and nothing in this section should be taken as a limitation on those claims. By way of introduction, the preferred embodiments described below include methods, computer readable media, instructions, and systems for determining temperature related information or detecting a temperature characteristic with a medical diagnostic ultrasound system. Anatomy information from an ultrasound scan is used with modeling to determine the temperature or other temperature related parameter. Ultrasound information may be obtained in real-time with application of thermal therapy, so may be used to better control heat generation and treatment. The anatomy information may be used to align model features measured from a region. The anatomy information may be used as an input into the model. The anatomy information may be used to select an appropriate model, such as selection based on the type of tissue. The anatomy information may be used to correct an output of the model, such as accounting for temperature distribution due to an adjacent vessel or other conductive tissue.

In a first aspect, a method of determining temperature related information with medical diagnostic ultrasound is provided. Ultrasound data representing anatomical information is acquired from a patient. Temperature related measurements are performed. The temperature related measurements are applied to a model. The model and the anatomical information are combined. The temperature related information is displayed as a function of the combining.

In a second aspect, a computer readable storage medium has stored therein data representing instructions executable by a programmed processor for detecting a temperature characteristic with a medical diagnostic ultrasound system. The storage medium includes instructions for: (a) receiving anatomical ultrasound information representing a patient, (b) during application of thermal therapy to a region of the patient, receiving ultrasound data representing different locations in the region, (c) modeling, with a time-dependent machine-trained model and during the application, a spatial distribution of temperature in the region as a function of the ultrasound data and a previous output of the modeling, the modeling responsive to the anatomical ultrasound information, and (d) outputting the spatial distribution of temperature.

In a third aspect, a system is provided for determining temperature related information with medical diagnostic ultrasound. A receive beamformer is configured to acquire ultrasound data representing a region of a patient. A processor is configured to model an effect of thermal therapy on the region with a machine-learned model and a thermal model. The machine-learned model uses at least one feature, which is a function of the ultrasound data. The thermal model is configured to correct an output of the machine-learned model as a function of the ultrasound data. A display is configured to display an image representing the effect.

Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the Figures, like reference numerals designate corresponding parts throughout the different views.

FIG. 1 is a flow chart diagram of one embodiment of a method for determining temperature related information with medical diagnostic ultrasound;

FIG. 2 is a graph of one example comparison of a modeled temperature with a thermocouple measured temperature;

FIG. 3 is an example medical ultrasound image with an overlaid temperature characteristic;

FIG. 4 is a three-dimensional rendering including temperature characteristic information according to one embodiment; and

FIG. 5 is a block diagram of one embodiment of a system for determining temperature related information with medical diagnostic ultrasound.

### DETAILED DESCRIPTION OF THE DRAWINGS AND PRESENTLY PREFERRED EMBODIMENTS

Target tissue temperatures are directly and non-invasively quantified and displayed in real-time using acoustic methods. Received ultrasound signals are interpreted. Acoustically-detected or other parameters are input into a model. For example, speed of sound, elasticity, backscatter intensity, and/or other information are used as features for a model. The model is a time dependent neural network, a piecewise linear fit in space and time, or other model. The model outputs measurements of temperature, thermal dose and/or other therapeutic tissue parameters useful in monitoring and/or controlling the treatment.

Ultrasound information may be combined with a temperature model in various ways. In some embodiments, anatomical ultrasound information is acquired. A therapeutic temperature related receive ultrasound parameter (e.g., speed of sound) is acquired. A time history of temperature, such as the historical output of the model during a therapy session, and the received ultrasound parameter are acquired over a period of time during treatment. The ultrasound parameter and the anatomical ultrasound information are combined with a model, such as a time dependent neural network or piecewise linear in space and time. The temperature measurement in one or more locations is derived from the model output.

In other embodiments, two or more temperature related ultrasound parameters, such as speed of sound, elasticity, ultrasound received signal de-correlation coefficient, and/or other parameters, are acquired. The parameters for one or more locations are input as features of the model. Anatomical information is combined with the model.

In other embodiments, one or more ultrasound parameters and therapeutic tissue parameters, such as dose, bioeffect, or tissue modification metrics, as well as the time history of these parameters over a period of time during treatment, are acquired. These parameters are input as features of the model, which is combined with anatomical information.

In another embodiment, non-ultrasound therapeutic parameters (e.g., temperature or dose) are acquired from sensors or a treatment device. The parameters are input in a thermal model to determine temperature distribution. The temperature distribution is registered to anatomic ultrasound information or otherwise combined with the anatomic information.

The use of modeling and anatomic information from ultrasound is provided during therapy. Non-real time embodiments are possible. Other embodiments are for training the model with a machine.

One or more combinations of anatomy information and models may be used. The anatomy information in raw form (e.g., B-mode or backscatter intensity data) or derived parameters (e.g., type of tissue) may be input as a feature of the model. The anatomy information may be used to align data for the same locations in the patient at different times, so that the model receives a correct time history of the parameter for each location. The anatomy information may be used to select a model, such as having different models for different types of tissue or body locations. The anatomy information may be used to correct an output of the model, such as modeling for uniform tissue and correcting the modeled output as a function of adjacent tissue (e.g., vessels, other heat sinks, or tissue inconsistencies).

Different types of models use the anatomy information. In one embodiment, the model is a machine-learned model with ultrasound-based features. In another embodiment, the temperature information is derived from any sensor (e.g., ultrasound, thermocouple, or other sensor) and the model is derived from theory or experimentation to predict temperature distribution from the input. In other embodiments, both types of models are used together, such as estimating temperature characteristics with a machine-learned model and using a thermal distribution model to correct for tissue or fluid variation.

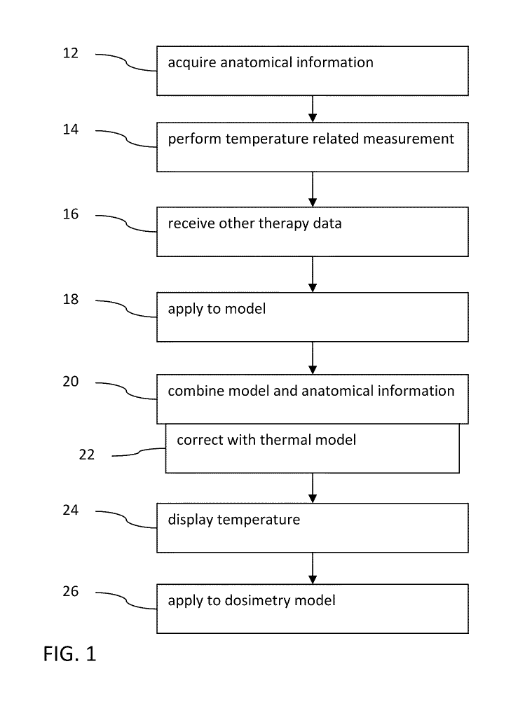

FIG. 1 shows one embodiment of a method of determining temperature related information with medical diagnostic ultrasound. This embodiment is directed to application of a model. In other embodiments, acts 12, 14, and/or 16 are performed for training a model. The acts are performed in the order shown or a different order. Additional, different, or fewer acts may be provided. For example, act 14 or 16 is optional. As another example, act 22 is performed as part of act 18 or is not provided. Acts 24 and 26 are optional.

The acts are performed during therapy. The acts are repeated throughout the therapy. For example, a reference set of data is acquired before application of the therapy. One or more parameters may be assumed for the initial iteration, such as assuming a temperature common for patients or type of tissue within a patient. Once thermal therapy begins, the acts are repeated to provide updated measurements and resulting model predictions. Changes in parameters may be used as input features with or without other parameters. A time history of the input parameters, the modeled output, or both may be used for modeling from any current measurement. The current estimated temperature, dose, or other temperature characteristic may be used to determine whether, where, and/or at what level to continue the therapy. In other embodiments, the temperature information is determined during later review.

In act 12, anatomical ultrasound information representing a patient is received. A medical diagnostic ultrasound system scans a region of the patient. Any type of scan, scan format, or imaging mode may be used. For example, harmonic imaging is used with or without added contrast agents. As another example, B-mode, color flow mode, spectral Doppler mode, M-mode, or other imaging mode is used.

Ultrasound data representing anatomical information is acquired from a patient. The ultrasound data represents a point, a line, an area, or a volume of the patient. Waveforms at ultrasound frequencies are transmitted, and echoes are received. The acoustic echoes are converted into electrical signals and beamformed to represent sampled locations within a region of the patient. The beamformed data may be filtered or otherwise processed. The beamformed data may be detected, such as determining an intensity. A sequence of echo signals from a same location may be used to estimate velocity, variance, and/or energy. Echoes at one or more harmonics of the transmitted waveforms may be processed. The detected values may be filtered and/or scan converted to a display format. The ultrasound data representing the patient is from any point along the ultrasound processing path, such as channel data prior to beamformation, radio frequency or in-phase and quadrature data prior to detection, detected data, or scan converted data.

The anatomical ultrasound information is the actual data. For example, B-mode data represents tissue structures. As another example, flow data indicates locations associated with a vessel. Alternatively or additionally, the anatomical ultrasound information is derived from the actual data. For example, a type of tissue at a given location is determined from a speckle characteristic, echo intensity, template matching with tissue structure, or other processing. As another example, region growing is used with B-mode data or color flow data to determine that the ultrasound data represents a vessel or other fluid region. A current distribution of anatomy, such as a list of represented organs, may be determined. The actual data and/or derived information are anatomical parameters to be used in combination with the model.

In act 14, temperature related measurements are performed. Any temperature related measurement may be used. For example, tissue expands when heated. Measuring the expansion may indicate temperature. Temperature related measurements may directly or indirectly indicate a temperature. For example, a measure of a parameter related to conductivity or water content (e.g., a measurement of the type of tissue) may indirectly impact the temperature. The measurements may be for raw ultrasound data or may be derived from ultrasound data.

Only one, or two or more measurements are performed. Measurements are performed for just one location, or for multiple locations in a region. Full or sparse sampling may be used. The measurements are performed over time, but independent of previous measurements. Alternatively or additionally, a change in a measurement from a reference or any previous (e.g., most recent) measurement may be used.

The temperature related measurements may use any modality. For example, a thermocouple, infrared, or other sensor is used. The sensor is inserted within the patient or scans the patient. As another example, information from the therapeutic treatment device is used. An energy output, dose, or other parameter of the thermal treatment is measured or received.

Non-real time measurements may be used, such as a baseline temperature. MRI-based measurements for temperature distribution in a region may be used. Real-time measurements may be used, such as associated with ultrasound measurements performed during application of thermal therapy to a region of the patient.

In one embodiment, one or more ultrasound measurements are performed with or without other temperature related measurements. Ultrasound measurements may be provided for a plurality of different locations in and/or around the treatment region. Any now known or later developed temperature related measurement using ultrasound may be used. In one embodiment, two or more, such as all four, of tissue displacement, speed of sound, backscatter intensity, and a normalized correlation coefficient of received signals are performed. Other measurements are possible, such as expansion of vessel walls.

Tissue displacement is measured by determining an offset in one, two, or three-dimensions. A displacement associated with a minimum sum of absolute differences or highest correlation is determined. The current scan data is translated, rotated, and/or scaled relative to a reference dataset, such as a previous or initial scan. The offset associated with a greatest or sufficient similarity is determined as the displacement. B-mode or harmonic mode data is used, but other data may be used. The displacement calculated for one location may be used to refine the search or search region in another location. Other measures of displacement may be used.

The speed of sound may be measured by comparison in receive time from prior to heating with receive time during heating. A pulse is transmitted. The time for the echo to return from a given location may be used to determine the speed of sound from the transducer to the location and back. Any aperture may be used, such as separately measuring for the same locations with different apertures and averaging. In another embodiment, signals are correlated. For example, in-phase and quadrature signals after beamformation are correlated with reference signals. A phase offset between the reference and current signals is determined. The frequency of the transmitted waveform (i.e., ultrasound frequency) is used to convert the phase difference to a time or speed of sound. Other measurements of the speed of sound may be used.

The backscatter intensity is B-mode or M-mode. The intensity or energy of the envelope of the echo signal is determined.

The normalized correlation coefficient of received signals may be measured. Beamformed data prior to detection, such as in-phase and quadrature data, is cross-correlated. In one embodiment, a reference sample or samples are acquired. During treatment, subsequent samples are acquired. For each location, a spatial window, such as three wavelengths in depth, defines the data for correlation. The window defines a length, area or volume. The current data is correlated with the reference data within the window space. The normalized cross-correlation is performed for the data in the window. As new data is acquired, further cross-correlation is performed.

Any temperature associated acoustic and physical parameters or changes in the parameters may be measured. Other measurements include tissue elasticity, strain, strain rate, motion (e.g., displacement or color flow measurement), or reflected power (e.g., backscatter cross-section). In act 16, other therapy data is received. The therapy data represents an aspect of the thermal therapy, such as an effect of the therapy. The effect may be temperature related or may merely be the result of application of heat beyond a particular dosage. The effect may persist after removal of the heat. Therapeutic effect- and bioeffect-associated parameters include elasticity (e.g., acoustic radiation force imaging), expansion (e.g., determined from B-mode tracking), shrinkage (e.g., determined from B-mode tracking), phase change, water content, flow or other fluid changes (e.g., coagulation determined from Doppler information) and other measurable changes. Changes in the therapy data parameters or history may be used.

Clinical or other information may be acquired. For example, genetic information or other tissue related data may be mined from a patient record. Any feature contributing to determination of temperature related information may be used.

The therapy data may be related to temperature. For example, expansion, shrinkage, water content, or other therapy parameters may indicate a current temperature. Regardless of the categorization of the measurement, the measurements are used as inputs to a model or to calculate values for input to the model. The therapy data is provided for one or more locations, such as providing therapy data for all locations in a two- or three-dimensional region. Alternatively, the therapy data is generally associated with the entire region, such as one dose or energy level for the entire region.

In act 18, the temperature related measurements and/or the therapy data are applied to a model. The measurements or data are input as raw data. Alternatively, the values (i.e., measurements and/or data) are processed and the processed values are input. For example, the values are filtered spatially and/or temporally. As another example, a different type of value may be calculated from the values, such as determining a variance, a derivative, normalized, or other function from the values. In another example, the change between the current values and reference or previous values is determined. A time-history of the values over a window of time may be used. The values are input as features of the model.

The output of the model may be used as an input. The values are applied during the application of thermal therapy. For an initial application of the model, the feedback is replaced with a reference temperature, such as the temperature of the patient. For further application of the model, the previous output is fed back as an input, providing a time-dependent model. The temperature related information output by the model is fed back as a time history of the information, such as temperature at one or more other times. During thermal therapy, the measured or received values are updated (i.e., current values are input for each application of the model), but previous values may also be used. The feedback provides an estimated spatial distribution of temperature or related information in the region at a previous time. The subsequent output of the model is a function of the ultrasound data or other values and a previous output of the modeling. The time-history of the values may be used as inputs, such that the time history and spatial distributions of the temperature-associated and therapeutic effect-related parameters are used as features of the model.

The model outputs a temperature or temperature distribution (i.e., temperature at different locations and/or times) from the input information. The derived temperature may be in any unit, such as degrees Fahrenheit or Celsius. The resolution of the temperature may be at any level, such as outputting temperature as in one of multiple three or other degree ranges. Alternatively, other temperature related information is output, such as a change in temperature, a dose, or an index value.

Any model may be used, such as a neural network or a piecewise linear model. The model is programmed or designed based on theory or experimentation. In one embodiment, the model is a machine-learned model. The model is trained from a set of training data labeled with a ground truth, such as training data associated with actual temperatures. For example, the various measures or receive data are acquired over time for each of multiple patients. During thermal therapy, the temperature is measured. The temperature is the ground truth. Through one or more various machine-learning processes, the model is trained to predict temperature given the values and/or any feedback.

Any machine-learning algorithm or approach to classification may be used. For example, a support vector machine (e.g., 2-norm SVM), linear regression, boosting network, probabilistic boosting tree, linear discriminant analysis, relevance vector machine, neural network, combinations thereof, or other now known or later developed machine learning is provided. The machine learning provides a matrix or other output. The matrix is derived from analysis of a database of training data with known results. The machine-learning algorithm determines the relationship of different inputs to the result. The learning may select only a sub-set of input features or may use all available input features. A programmer may influence or control which input features to use or other performance of the training. For example, the programmer may limit the available features to measurements available in real-time. The matrix associates input features with outcomes, providing a model for classifying. Machine training provides relationships using one or more input variables with outcome, allowing for verification or creation of interrelationships not easily performed manually.

The model represents a probability of temperature related information. This probability is a likelihood for the temperature related information. A range of probabilities associated with different temperatures is output. Alternatively, the temperature with the highest probability is output. In other embodiments, the temperature related information is output without probability information.

As an alternative to machine learning, manually programmed models may be used. The model may be validated using machine training. In one embodiment, a thermal distribution model is used. The thermal distribution model accounts for the thermal conductivity, density, or other behavior of different tissues, fluids, or structures. The thermal distribution model receives temperatures, temperature related information, measurements, or other data. The input information may be sparse, such as having temperature information for one or more, but fewer than all locations. The thermal distribution model determines the temperature at other locations. The thermal distribution model may determine the temperature at other times or both time and location.

In another embodiment, the thermal distribution model corrects temperatures based on anatomy. For example, a machine-learned model estimates temperature for uniform tissue. The temperature output is corrected to account for tissue differences in the region, such as reducing the temperature around thermally conductive vessels or fluid regions.

In act 20, the model and the anatomical information from act 12 are combined. The modeling and output temperature related information are responsive to the anatomical ultrasound information. Any function may be used. Different combination embodiments are provided below, but other embodiments may be used. Two or more combinations may be used together.

In one embodiment, the ultrasound data are spatially aligned. Any features associated with a spatial distribution or location may be aligned or registered based on the anatomy. Tissue, the patient, and/or the transducer may move during scanning. As a result, measurements at a particular location in one scan may be for a different location in another scan. The anatomy information is used to align the measurements so that data for the appropriate locations is identified. For example, the data from a center of a treatment region is aligned through different scans.

The alignment is performed prior to input to the model or prior to application of the model in act 18. The alignment avoids data from other locations providing a value not appropriate for a given location.

Any alignment may be used. A position sensor on the transducer may be used to correct for transducer movement. Data correlation along one, two, or three dimensions may be used to account for any source of motion. The anatomical ultrasound information represents the tissue structure, such as a B-mode image representing the region. The anatomical information is acquired for each time period, such as associated with each measurement for a parameter or set of parameters. The anatomical information spatially corresponds with the measurements taken at substantially the same time. Substantially accounts for interleaving different types of ultrasound transmissions and receptions. The B-mode data from the different times is aligned by identifying a translation, scale, and/or rotation associated with a minimum sum of absolute differences, a highest correlation, or other similarity measure. The anatomical data represents the entire region. Alternatively, separate alignments are performed for each location or for sub-sections of the region. Surrounding data for a given location may be used to determine the match. The translation, scale, and/or rotation with the greatest similarity are applied to shift the locations associated with the measurements.

In another embodiment, the anatomical information is combined with the modeling by selection. The anatomical information indicates the type of tissue or anatomy being treated. Anatomic information may indicate blood vessel proximity as a heat sink, tissue type influencing thermal characteristics, or parameters in a bio-heat equation (e.g., specific heat, conductivity, and density). The type of tissue is determined by either operators or automated classification algorithms applied to the ultrasonic and its derived data. The existence of blood vessels may be indicated by fluid pools represented by color flow imaging. Template matching may be used to identify the region from B-mode or other ultrasound data, and knowledge of the specific region provides information for selection.

Different models are provided for different types of tissue. Alternatively or additionally, different models are provided for different regions of a patient. The anatomy information is used to select the appropriate model for a given treatment region. Other criteria for the selection of the model may also be used. More than one model may be used. Different models or one model may apply to different spatial locations, tissue types, or temperature ranges.

Some models may be suitable for specific situations. For example, a patient being treated may have a temperature sensor inserted into or adjacent to a tumor. A model trained based on training data where such temperature sensors are provided is selected. The anatomy information may indicate the existence of the sensor. Alternatively, the user indicates one or more variables used to select the appropriate model.

In another embodiment, the modeling is combined with the anatomical information by correction of spatial and/or temporal distribution of temperature related information in act 22. For example, the thermal distribution model is applied to the temperature output of a machine-trained model. The anatomical information is used to select or create the thermal model. The type of tissue or fluid and locations are determined. The thermal distribution model accounting for the types of tissue and relative locations is applied to the temperature output. The output of the thermal distribution model corrects the output of the machine-learned model.

In another embodiment, the thermal distribution model is used without output from another model. The input information is sparse, such as a temperature in time and/or location less than all times or locations. For example, thermal sensors on a treatment device (e.g., on a radio frequency ablation needle) or separately inserted in the patient (e.g., a thermocouple probe) provide measurements of temperature during treatment. The thermal distribution model is used to determine the temperature at other times or locations. The anatomical information is used to model the thermal distribution, such as determining conductivity as a function of location based on tissue type. The thermal distribution model, based on the anatomy, determines the temperature in space and/or time.

In another embodiment, the anatomical information is applied as a feature to the model in act 18. The anatomical information, such as the type of tissue by location, existence of fluid regions in the treatment zone, or other anatomical information, is input to the model.

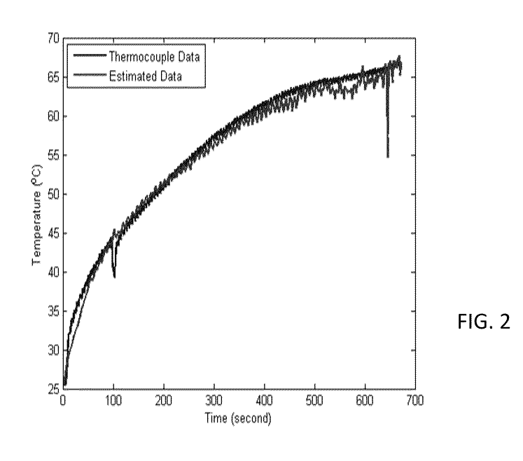

In response to input of the features, the model outputs the temperature related information, such as temperature. FIG. 2 shows an example output from a machine trained neural network. The model used displacement in two-dimensions, elasticity in two-dimensions, normalized cross-correlation coefficient in two-dimensions, and backscatter intensity in two-dimensions as input features. The darker, more consistent (i.e., less variable) line, represents temperature measured by a thermocouple during thermal treatment. The lighter, more variable line represents the model output averaged over a 2.5 mm by 2.5 mm region of interest.

In act 24, the temperature related information is displayed. The temperature related information is based, at least in part, on the combination of modeling and anatomical ultrasound information. The temperature related information is displayed as a value, such as a temperature or dose. A graph of temperature as a function of time or along a line may be displayed.

In one embodiment, the temperature is mapped to color and overlaid on a two-dimensional image or a three-dimensional representation. The mapping modulates the color as a function of the temperature related information, such as the shade of red or color between red and yellow being different for different temperatures. The change in temperature may alternatively be mapped to the output color or additionally mapped to brightness or other aspect of the color. The overlay is laid over an ultrasound image representing the anatomy, such as overlaid on a B-mode image. The overlay is registered to the anatomic information.

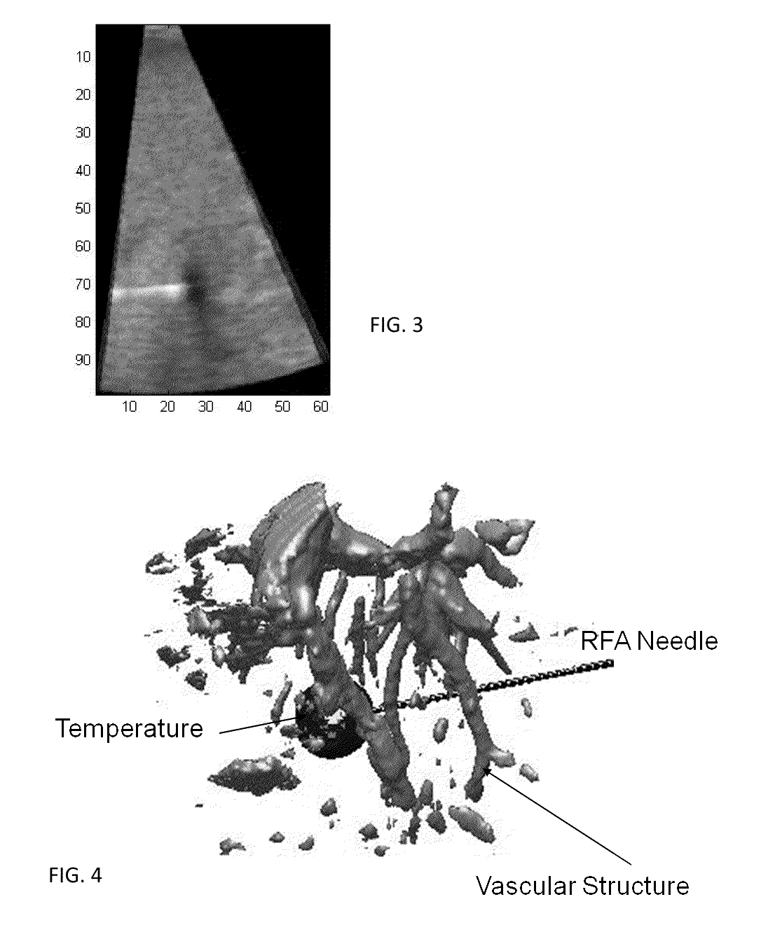

The spatial distribution of the temperature or related information is represented on overlay of the image. A separate temperature image may be generated. The temperature at different locations is indicated. FIG. 3 shows B-mode ultrasound information with a color overlay (shown in grayscale). The region in the lower center of the image is darker due to the color overlay. The darkest region corresponds to the highest temperature. FIG. 3 represents the detected or estimated temperature at the end-of-dose or therapy.

FIG. 4 represents a three-dimensional rendering from color flow information. The vasculature structure is shown. A radio frequency ablation needle representation is added, based on B-mode return or position sensing. At the end of the needle is a spherical region represented in two-dimensions as an oval. The spherical region is mapped from temperature information. The proximity of the vascular structure is used to correct the temperature information prior to mapping.

The images are provided in real-time or as acquired. The needle is within the patient, ablating tissue. The image shows the resulting temperature distribution, providing an indication of the therapeutic effect registered and overlaid on the anatomic information.

The temperature related information may be further processed. Act 26 shows one example. The temperature related information is applied to a dosimetry model. The dosimetry model determines the thermal dose, such as the maximum thermal dose in the region, an average or overall dose, or the thermal dosage for different locations. The thermal dose is determined from an amount of time and temperature, but may be based on other factors. The temperatures at different locations are used to determine the dose at the different locations or an overall dose for the regions, such as an average or total dose. Any now known or later developed dosimetry model may be used, such as Saparetto-Dewey, a dosimetry equation, or cumulative equivalent minutes at a reference temperature. The dosimetry model outputs dose.

The dose information is displayed as a number, graph, or image. For example, spatially distributed dose information is registered to and overlaid as a color on anatomic image, such as a B-mode image.

Based on the temperature, dose, and/or other temperature related information, the therapy may be controlled. The control is manual, such as the user selecting adjustments or an end point for thermal therapy based on the temperature related information. Alternatively, the control is automatic, such as ceasing or varying therapy when a temperature and/or dose are reached. In other embodiments, the temperature information from during the therapy or at an end of therapy is used to determine a prognosis or therapy result at a later time.

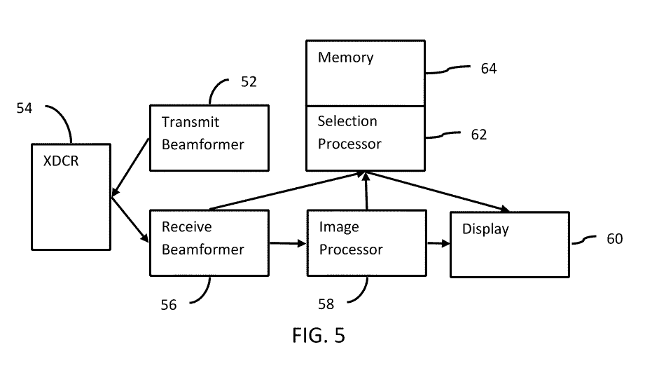

FIG. 5 shows one embodiment of a system for determining temperature related information with medical diagnostic ultrasound. The system performs the method described above or a different method. Other systems may be used. The ultrasound system includes a transmit beamformer 52, a transducer 54, a receive beamformer 56, an image processor 58, a display 60, a processor 62 and a memory 64. Additional, different or fewer components may be provided. For example, separate detectors and scan converter are also provided. As another example, a separate therapy transducer or treatment system is provided.

The system 10 is a medical diagnostic ultrasound imaging system. Imaging includes two-dimensional, three-dimensional, B-mode, Doppler, color flow, spectral Doppler, M-mode or other imaging modalities now known or later developed. The ultrasound system 10 is a full size cart mounted system, a smaller portable system, a hand-held system or other now known or later developed ultrasound imaging system. In another embodiment, the processor 62 and memory 64 are part of a separate system. For example, the processor 62 and the memory 64 are a workstation or personal computer operating independently of the ultrasound system. As another example, the processor 62 and the memory 64 are part of a therapy system.

The transducer 54 comprises a single, one-dimensional, multi-dimensional or other now known or later developed ultrasound transducer. Each element of the transducer 54 is a piezoelectric, microelectromechanical, capacitive membrane ultrasound transducer, or other now known or later developed transduction element for converting between acoustic and electrical energy. Each of the transducer elements connect to the beamformers 52, 56 for receiving electrical energy from the transmit beamformer 52 and providing electrical energy responsive to acoustic echoes to the receive beamformer 56.

The transmit beamformer 12 is one or more waveform generators, amplifiers, delays, phase rotators, multipliers, summers, digital-to-analog converters, filters, combinations thereof and other now known or later developed transmit beamformer components. The transmit beamformer 52 is configured into a plurality of channels for generating transmit signals for each element of a transmit aperture. The transmit signals for each elements are delayed and apodized relative to each other for focusing acoustic energy along one or more scan lines. Signals of different amplitudes, frequencies, bandwidths, delays, spectral energy distributions or other characteristics are generated for one or more elements during a transmit event.

The receive beamformer 56 is configured to acquire ultrasound data representing a region of a patient. The ultrasound data is for measuring temperature related information, acquiring anatomical information, and/or receiving other therapy data. The anatomical information is, at least in part, from ultrasound data. The model uses none, one or more input features from ultrasound data. Other sources of data include sensors, a therapy system, or other inputs. Such devices or inputs may be provided to the processor 62 or the memory 64. In one embodiment, all of the inputs features used by the model and the anatomical information are acquired from ultrasound data.

The receive beamformer 56 includes a plurality of channels for separately processing signals received from different elements of the transducer 54. Each channel may include delays, phase rotators, amplifiers, filters, multipliers, summers, analog-to-digital converters, control processors, combinations thereof and other now known or later developed receive beamformer components. The receive beamformer 56 also includes one or more summers for combining signals from different channels into a beamformed signal. A subsequent filter may also be provided. Other now known or later developed receive beamformers may be used. Electrical signals representing the acoustic echoes from a transmit event are passed to the channels of the receive beamformer 56. The receiver beamformer outputs in-phase and quadrature, radio frequency or other data representing one or more locations in a scanned region. The channel data or receive beamformed data prior to detection may be used by the processor 62.

The receive beamformed signals are subsequently detected and used to generate an ultrasound image by the image processor 58. The image processor 58 is a B-mode/M-mode detector, Doppler/flow/tissue motion estimator, harmonic detector, contrast agent detector, spectral Doppler estimator, combinations thereof, or other now known or later developed device for generating an image from received signals. The image processor 58 may include a scan converter. The detected or estimated signals, prior to or after scan conversion, may be used by the processor 62.

The display 60 is a monitor, LCD, plasma, projector, printer, or other now known or later developed display device. The display 60 is configured to display an image representing the effect of thermal therapy. For example, the temperature or related information is output as a value, graph, or two-dimensional representation. The processor 62 and/or the image processor 58 generate display signals for the display 60. The display signals, such as RGB values, may be used by the processor 62.

The processor 62 is a control processor, beamformer processor, general processor, application specific integrated circuit, field programmable gate array, digital components, analog components, hardware circuit, combinations thereof and other now known or later developed devices for processing information. The processor 62 is configured, with computer code, to model an effect of thermal therapy on a treatment region. For example, the temperature for one or more locations in the treatment region is estimated based on inputs. The computer code implements a machine-learned model and/or a thermal model to estimate the temperature or temperature related information. The model is a matrix, algorithm, or combinations thereof to estimate based on one or more input features.

The processor 62 receives, requests, and/or calculates values for the features input to the model. In one embodiment, one or more of the features and corresponding values are a function of the ultrasound data. A single value is provided for each feature for the region. Multiple values per feature may be applied to represent the feature at different times and/or locations. The values of the feature are from raw data, such as B-mode values, or are calculated, such as using tracking or correlation.

The processor 62 applies the values for a current time or model application. The values are of current measures, previous measures, or changes between measures. In one embodiment, one or more of the features are previous outputs of the modeling. A time-dependent model is configured to model the effect as a function of a previous output of the time-dependent model. An initial input may be an assumed value, such as 37 degrees Celsius, or a reference measurement before the start of therapy. The time-dependent model and feedback may be used during application of the thermal therapy. The trend or change is accounted for by the feedback, allowing for predictive control of the thermal therapy. The feedback is of the raw output or is calculated from the previous output or outputs, such as a feature for a change in temperature over a given time period.

In another embodiment, the processor 62 is configured to implement a thermal model. Using thermal calculations for different tissues, such as based on conductivity, density, and an applied dose or current estimated temperature, the spatial and/or temporal distribution of temperature related information is determined. For example, a thermal model corrects an output of another model, such as correction of temperature output by a machine-learned model. The machine-learned model may assume structure associated with a particular anatomy or assume uniform tissue of one type. Ultrasound anatomical information is used to determine tissues and fluids in a region, allowing for correction of temperatures based on thermal conductivity and/or density.

The memory 64 is a computer readable storage medium having stored therein data representing instructions executable by the programmed processor for detecting a temperature characteristic with a medical diagnostic ultrasound system. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method of determining temperature related information with medical diagnostic ultrasound, the method comprising:
acquiring ultrasound data representing anatomical information from a patient;
performing temperature related measurements;
applying the temperature related measurements to a model;
combining the model and the anatomical information; and
displaying the temperature related information as a function of the combining.

2. The method of claim 1 wherein acquiring comprises acquiring the ultrasound data representing a type of tissue, further comprising determining the type of tissue.

3. The method of claim 1 wherein acquiring comprises acquiring the ultrasound data representing fluid region.

4. The method of claim 1 wherein acquiring comprises acquiring the ultrasound data representing anatomical distribution.

5. The method of claim 1 wherein performing temperature related measurements comprises determining the temperature related measurements from a therapeutic treatment device, sensor, or both the sensor and the therapeutic treatment device.

6. The method of claim 1 wherein performing temperature related measurements comprises performing one or more ultrasound measurements.

7. The method of claim 6 wherein performing the one or more ultrasound measurements comprises performing at least two of: tissue displacement, speed of sound, backscatter intensity, and a normalized correlation coefficient of received signals.

8. The method of claim 1 wherein applying comprises applying with the model comprising a machine-learned neural network model, the temperature related measurements being input to the machine-learned neural network model, and the machine-learned neural network model outputting a temperature.

9. The method of claim 1 wherein applying comprises applying a thermal distribution model, the temperature related measurements being for fewer locations, times or both locations and times than output by the thermal distribution model.

10. The method of claim 1 wherein combining comprises selecting the model from a group of models based, at least in part, on the anatomical information.

11. The method of claim 1 wherein combining comprises aligning the temperature related information from different times and prior to applying, the aligning being performed as a function of the anatomical information.

12. The method of claim 1 wherein combining comprises applying the anatomical information to the model.

13. The method of claim 1 wherein combining comprises correcting an output of the model with a thermal model, the thermal model receiving the anatomical information and the output of the model.

14. The method of claim 1 wherein displaying comprises displaying a color overlay of an ultrasound image representing anatomy of the patient, the color overlay having colors modulated as a function of the temperature related information.

15. The method of claim 1 further comprising applying the temperature related information to a dosimetry model.

16. The method of claim 1 further comprising feeding back the temperature related information, as a time history of temperature, to the model, the model receiving as inputs the time history of temperature and the temperature related measurements during application of thermal energy-based treatment to the patient.

17. In a computer readable storage medium having stored therein data representing instructions executable by a programmed processor for detecting a temperature characteristic with a medical ultrasound system, the storage medium comprising instructions for:
receiving anatomical ultrasound information representing a patient;
during application of thermal therapy to a region of the patient, receiving ultrasound data representing different locations in the region;
modeling, with a time-dependent machine-trained model and during the application, a spatial distribution of temperature in the region as a function of the ultrasound data and a previous output of the modeling, the modeling responsive to the anatomical ultrasound information; and
outputting the spatial distribution of temperature.

18. The computer readable storage medium of claim 17 wherein modeling responsive to the anatomical ultrasound information comprises:
(a) spatially aligning the ultrasound data prior to input to the modeling, the spatially aligning being based on correlation of the anatomical ultrasound information;
(b) selecting the time-dependent machine-trained model based on a type of tissue or anatomy represented by the anatomical ultrasound information;
(c) correcting the spatial distribution of temperature as a function of the anatomical ultrasound information; or
(d) combinations thereof.

19. The computer readable storage medium of claim 17:
further comprising receiving therapy data representing an aspect of the thermal therapy;
wherein modeling comprises modeling as a function of the therapy data; and
wherein outputting comprises outputting information representing the spatial distribution of temperature or dose determined as a function of the spatial distribution of temperature.

20. A system for determining temperature related information with medical diagnostic ultrasound, the system comprising:
a receive beamformer configured to acquire ultrasound data representing a region of a patient; and
a processor configured to model an effect of thermal therapy on the region with a machine-learned model and a thermal model, the machine-learned model using at least one feature, which is a function of the ultrasound data, the thermal model configured to correct an output of the machine-learned model as a function of the ultrasound data.

21. The system of claim 20 wherein the machine-trained model comprises a time-dependent model configured to model the effect during application of the thermal therapy and as a function of a previous output of the time-dependent model during the application.

22. The system of claim 20 further comprising:
a display configured to display an image representing the effect.
